(19) United States
(12) Patent Application Publication
(10) Pub. No.: US 2021/0128155 A1
(43) Pub. Date: May 6, 2021

(54) SURGICAL CLIP APPLIER WITH PARTIAL CLAMPING

(71) Applicant: Covidien LP, Mansfield, MA (US)

(72) Inventors: Jacob C. Baril, Norwalk, CT (US); Matthew A. Dinino, Newington, CT (US); Justin Thomas, New Haven, CT (US); Roy J. Pilletere, North Haven, CT (US)

(21) Appl. No.: 17/070,440

(22) Filed: Oct. 14, 2020

Related U.S. Application Data
(60) Provisional application No. 62/930,767, filed on Nov. 5, 2019.

(51) Int. Cl. A61B 17/128; A61B 17/28

(52) U.S. Cl. CPC A61B 17/128; A61B 2017/00734; A61B 17/2841

(57) ABSTRACT

A surgical clip applier includes a handle assembly including an actuator and a motor, an elongate body including a drive member, and a jaw assembly that receives a clip. The actuator is operable to activate the motor to move the jaw assembly to partially crimp a clip or fully crimp a clip.

### CROSS-REFERENCE TO RELATED APPLICATION

This application claims the benefit of and priority to U.S. Provisional Patent Application No. 62/930,767 filed Nov. 5, 2019, the entire disclosure of which is incorporated by reference herein.

### FIELD

The disclosure is directed to surgical clip appliers and, more particularly to motor driven surgical clip appliers.

### BACKGROUND

Surgical clip appliers for ligating tissue are well known in the surgical arts. These clip appliers are available in both open and endoscopic configurations and typically include a handle assembly, an elongate body, and a jaw assembly. The handle assembly includes an actuator and a drive member that extends from the handle assembly through the elongate body to the jaw assembly. The elongate body supports a cartridge assembly that includes a plurality of clips that are sequentially fed to the jaw assembly in response to movement of the drive member.

In known clip appliers, upon operation of the actuator, the drive member is driven a fixed longitudinal distance to crimp one of the clips to a predetermined degree. It is not always desirable to fully crimp each clip. For example, in situations where tissue is thick, fully crimping a clip may damage the tissue. In addition, in certain surgical procedures such as cholangiograms in which a catheter is secured to a vessel using a clip, fully crimping the clip can occlude the vessel and prevent contrast media from entering the vessel.

A continuing need exists in the art for a clip applier that can be selectively operated to partially crimp a clip.

### SUMMARY

One aspect of this disclosure is directed to a surgical clip applier including a handle assembly, an elongate body, and a jaw assembly. The handle assembly includes a housing defining a cavity, an actuator and a motor positioned within the cavity of the housing. The elongate body includes an outer tubular housing and a drive member movable within the outer tubular housing. The drive member has a proximal portion that extends into the cavity of the housing and a distal portion. The proximal portion of the drive member is coupled to the motor, and the drive member is movable from retracted position to an advanced position in response to activation of the motor. The jaw assembly includes first and second jaw members that are configured to receive and support a clip. The first and second jaw members are movable from an open position to a clamped position and are operably engaged with the distal portion of the drive member such that movement of the drive member from the retracted position to the advanced position moves the first and second jaw members from the open position to a fully clamped position to fully crimp a clip. The actuator is configured to activate the motor to selectively advance the drive member to a predetermined partially advanced position between the retracted and advanced positions to move the first and second jaw members to a partially clamped position between the open and clamped positions to partially crimp the clip positioned between the first and second jaw members.

In aspects of the disclosure, the handle assembly includes a link and the motor includes a rotatable drum, wherein the drive member is coupled to the rotatable drum of the motor by the link such that rotation of the rotatable drum causes movement of the drive member between the retracted and advanced positions.

In some aspects of the disclosure, the handle assembly includes a controller circuit that is coupled to the motor and the actuator and is configured to control operation of the motor to selectively advance the drive member to either the advanced position or the partially advanced position.

In certain aspects of the disclosure, the controller circuit includes one or more integrated circuits or chips.

In aspects of the disclosure, the handle assembly includes a battery that is coupled to the motor via the controller circuit.

In some aspects of the disclosure, the housing of the handle assembly includes a grip portion and the battery is received in a battery pack that is releasably coupled to the grip portion.

In certain aspects of the disclosure, the actuator includes first and second actuation buttons coupled to the motor, wherein the first actuator button is operable to activate the motor to move the drive member to the partially advanced position to partially crimp the clip and the second actuator button is operable to activate the motor to move the drive member to the advanced position to fully crimp the clip.

In aspects of the disclosure, the controller circuit is configured to control advancement of the drive member to any desired longitudinal position between the advanced and retracted positions to cause any desired degree of crimping of the clip.

In some aspects of the disclosure, each of the first and second jaw members defines a channel that receives the clip.

In certain aspects of the disclosure, the surgical clip applier includes a rotation knob that supports the elongate body, wherein the rotation knob is rotatably coupled to the housing of the handle assembly and is rotatable in relation to the handle assembly to facilitate rotation of the elongate body and jaw assembly in relation to the handle assembly.

Another aspect of the disclosure is directed to a surgical clip applier that includes a handle assembly, an elongate body, and a jaw assembly. The handle assembly includes an actuator and a motor. The elongate body is supported on the handle assembly and includes a drive member having a proximal portion coupled to the motor and a distal portion. The jaw assembly includes first and second jaw members that are operably engaged with the drive member and movable in relation to each other in response to movement of the drive member between open and clamped positions. A clip is supported between the first and second jaw members. The actuator includes a first actuator that is operable to move the drive member to a first position to move the first and second jaw members to a first jaw member position between the open and clamped positions to partially crimp a clip and a second actuator that is operable to advance the drive member to the advanced position to move the first and second jaw members to the clamped position to fully crimp the clip.

Yet another aspect of the disclosure is directed to a surgical clip applier including a handle assembly, an elongate body, and a jaw assembly. The handle assembly includes a housing defining a cavity, an actuator, a motor, a link, a controller circuit coupled to the actuator and the motor, and a battery coupled to the motor via the controller circuit. The motor and the controller circuit are positioned within the cavity of the housing and the actuator includes a first actuator and a second actuator. The elongate body includes an outer tubular housing and a drive member movable within the outer tubular housing. The drive member has a proximal portion that extends into the cavity of the housing and a distal portion. The proximal portion is coupled to the motor by the link. The drive member is movable from a retracted position to an advanced position in response to activation of the motor. The jaw assembly includes first and second jaw members that are configured to receive and support a clip. The first and second jaw members are movable in relation to each other from an open position to a clamped position and are operably engaged with the distal portion of the drive member such that movement of the drive member from the retracted position to the advanced position moves the first and second jaw members from the open position to a fully clamped position. The first actuator is operable to move the drive member to a first position to move the first and second jaw members to a first jaw member position between the open and clamped positions to partially crimp the clip and the second actuator that is operable to advance the drive member to the advanced position to move the first and second jaw members to the clamped position to fully crimp the clip.

Other features of the disclosure will be appreciated from the following description.

### BRIEF DESCRIPTION OF THE DRAWINGS

Various aspects of the disclosed surgical clip applier are described herein below with reference to the drawings, wherein:

FIG. 1 is a side perspective view of exemplary aspects of the disclosed surgical clip applier;

FIG. 2 is a side perspective view of a distal portion of the surgical clip applier shown in FIG. 1 illustrating a distal portion of the elongate body and a jaw assembly with the jaw assembly in an open position and an uncrimped clip positioned between the jaw members of the jaw assembly;

FIG. 3 is a side perspective view of a clip of the surgical clip applier shown in FIG. 1;

FIG. 4 is an exploded, side perspective view of the handle assembly and elongate body of the surgical clip applier shown in FIG. 2 with the rotation knob supported about the portion of the elongate body;

FIG. 5 is a side view of the proximal portion of the surgical clip applier shown in FIG. 1 illustrating a handle assembly and a portion of an elongate body of the surgical clip applier with a portion of a handle housing and a rotation knob removed;

FIG. 6 is a side perspective view of a proximal portion of the surgical clip applier shown in FIG. 1 illustrating a handle assembly and a proximal portion of an elongate body of the surgical clip applier with a portion of a handle housing and a rotation knob removed and a drive member in a retracted position;

FIG. 7 is a side view of the proximal portion of the surgical clip applier shown in FIG. 1 illustrating the handle assembly and the proximal portion of the elongate body of the surgical clip applier with a portion of the handle housing and the rotation knob removed and the drive member in a partially advanced position;

FIG. 8 is a side perspective view of the distal portion of the surgical clip applier shown in FIG. 5 illustrating the distal portion of the elongate body and the jaw assembly with the jaw assembly in a partially clamped position and a clip partially crimped between the jaw members of the jaw assembly;

FIG. 8A is a side perspective view of the distal portion of the surgical clip applier shown in FIG. 8 with a portion of an outer sleeve of the elongate body cutaway illustrating the drive member in a partially advanced position, the jaw assembly in the partially clamped position, and the clip in a partially crimped position;

FIG. 9 is a side perspective view of the partially crimped clip shown in FIG. 8A;

FIG. 10 is a side view of the proximal portion of the surgical clip applier shown in FIG. 1 illustrating the handle assembly and the proximal portion of the elongate body of the surgical clip applier with a portion of the handle housing and the rotation knob removed and the drive member in a fully advanced position;

FIG. 11 is a side perspective view of the distal portion of the surgical clip applier shown in FIG. 5 illustrating the distal portion of the elongate body and the jaw assembly with the jaw assembly in a fully clamped position and the clip fully crimped between the jaw members of the jaw assembly;

FIG. 11A is a side perspective view of the distal portion of the surgical clip applier shown in FIG. 11 with a portion of the outer sleeve of the elongate body cutaway illustrating the drive member in a fully advanced position, the jaw assembly in the fully clamped position, and the clip in a fully crimped position;

FIG. 12 is a side perspective view of the fully crimped clip shown in FIG. 11 positioned about tissue;

FIG. 13 is a side view of other exemplary aspects of the disclosed surgical clip applier illustrating the handle assembly including a removable battery pack; and

FIG. 14 is a side view of a portion of the handle assembly shown in FIG. 13 with the battery pack removed from a remaining portion of the handle assembly.

### DETAILED DESCRIPTION

The disclosed surgical clip applier will now be described in detail with reference to the drawings in which like reference numerals designate identical or corresponding elements in each of the several views. However, it is to be understood that the aspects of the disclosure described herein are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure.

In this description, the term “proximal” is used generally to refer to that portion of the device that is closer to a clinician, while the term “distal” is used generally to refer to that portion of the device that is farther from the clinician. In addition, the term “endoscopic” is used generally to refer to endoscopic, laparoscopic, arthroscopic, and/or any other procedure conducted through small diameter incision or cannula. Further, the term “clinician” is used generally to refer to medical personnel including doctors, nurses, and support personnel.

The disclosed surgical clip applier includes a handle assembly, an elongate body, and a jaw assembly. The handle assembly includes a motor and a battery pack coupled to the motor for powering the motor. The elongate body includes a drive member having a proximal portion and a distal portion. The proximal portion is coupled to the motor of the handle assembly and the distal portion is engaged with the jaw assembly. The elongate body supports a plurality of clips that are sequentially advanced into the jaw assembly. The jaw assembly is engaged with the drive member and is movable between an open position and a fully clamped position in response to longitudinal movement of the drive member. The motor can be actuated to advance the drive member to at least two different longitudinal positions within the elongate body to move the jaw assembly from the open position to at least one partially clamped position and to the fully clamped position to selectively crimp one of the plurality of clips to a desired degree.

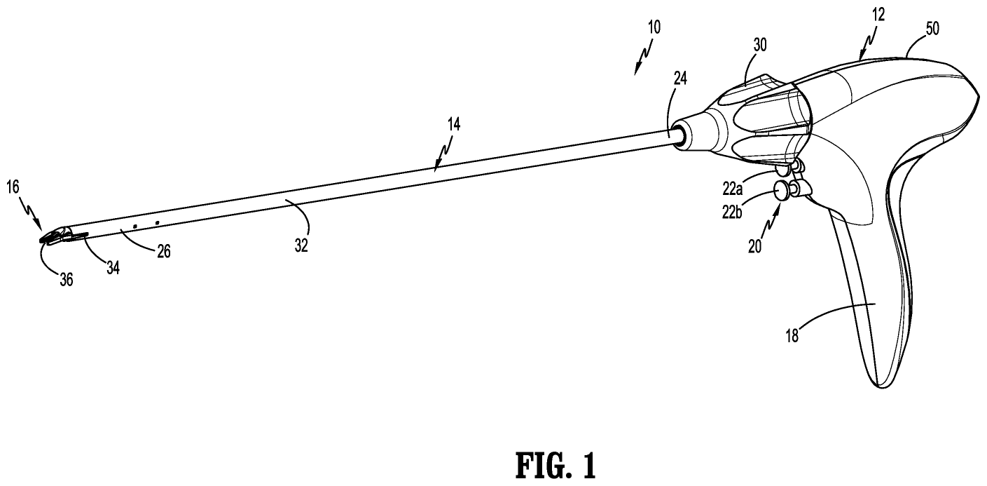

FIG. 1 illustrates the disclosed surgical clip applier shown generally as clip applier 10. The clip applier 10 includes a handle assembly 12, an elongate body 14, and a jaw assembly 16. The handle assembly includes a grip portion 18, and an actuator 20. In aspects of the disclosure, the actuator 20 includes a plurality of actuation buttons 22a, 22b that can be depressed to operate the clip applier 10 as described in detail below. It is envisioned that the actuator 20 may assume a variety of configurations and need not be in the form of buttons 22a, 22b. The elongate body 14 includes a proximal end 24 and a distal end 26 and defines a longitudinal axis “X”. The proximal end 24 is supported by the handle assembly 12. In certain aspects of the disclosure, the proximal end 24 of the elongate body 14 is coupled to the handle assembly 12 by a rotation knob 30. The rotation knob 30 is supported on the handle assembly 12 and supports the elongate body 14 such that rotation of the rotation knob 30 in relation to the handle assembly causes corresponding rotation of the elongate body 14 about the longitudinal axis “X”. The elongate body 14 includes an outer tubular housing 32 that houses internal components of the elongate body 14. In aspects of the disclosure, the outer tubular housing 32 includes a distal portion that defines elongate slots 34 that extend through the distal end of the distal portion of the elongate body 14.

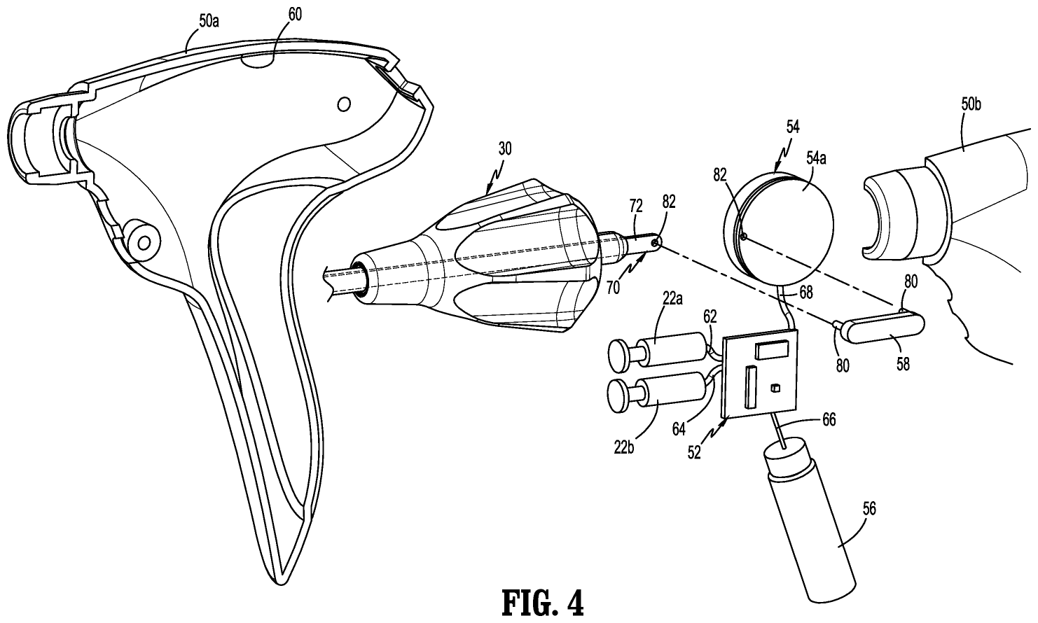

The elongate body 14 supports a staple cartridge (not shown) that includes a plurality of clips 36 (FIG. 4). The clips 36 are longitudinally aligned within the staple cartridge and are sequentially fed to the jaw assembly 16 as the clip applier 10 is repeatedly actuated. For a more detailed description of the internal components of the elongate body, see, e.g., U.S. Pat. Nos. 8,894,665, 8,382,773, and 7,905,890.

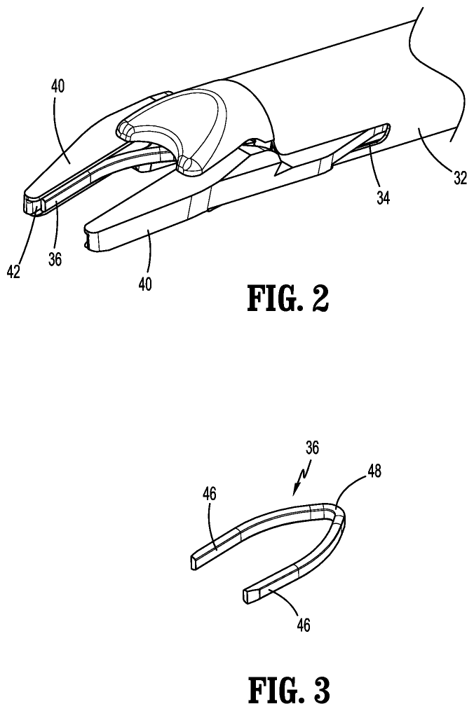

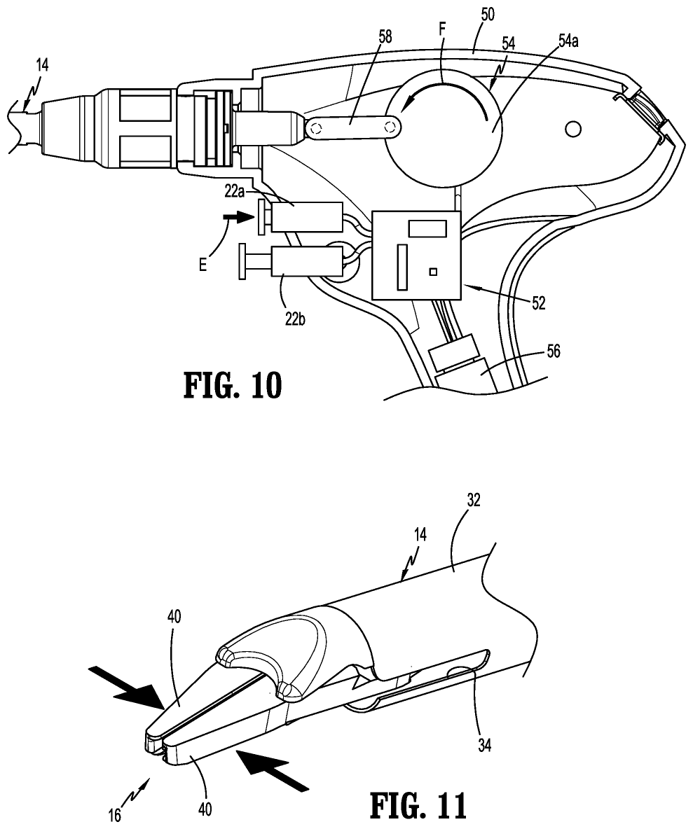

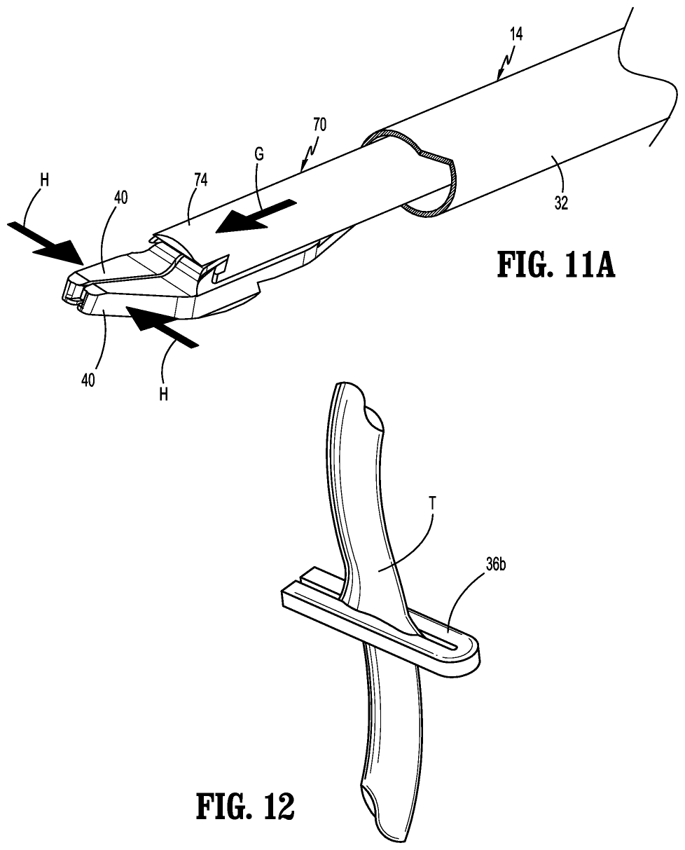

FIGS. 2 and 3 illustrate the distal portion of the clip applier 10 including the jaw assembly 16 in an open position and a clip 36 received between spaced jaw members 40 of the jaw assembly 16. The jaw assembly 16 is supported within and extends from a distal portion of the elongate body 14. The jaw members 40 define longitudinal channels 42 that sequentially receive the clip 36 from within the elongate body 14 as the clip applier 10 is actuated. The jaw members 40 are formed of a resilient material such as spring steel and are movable between the open position (FIG. 2) and a fully clamped position (FIG. 11) to crimp or partially crimp the clip 36 positioned between the jaw members 40. In the open position, the jaw members 40 project through the elongate slots 34 of the outer tubular housing 32 of the elongate body 14.

In aspects of the disclosure, each of the clips 36 is substantially U-shaped or V-shaped and includes first and second legs 46 and a back span 48. The clips 36 can be formed from titanium or other suitable material deformable material that can be safely received within the body of a patient.

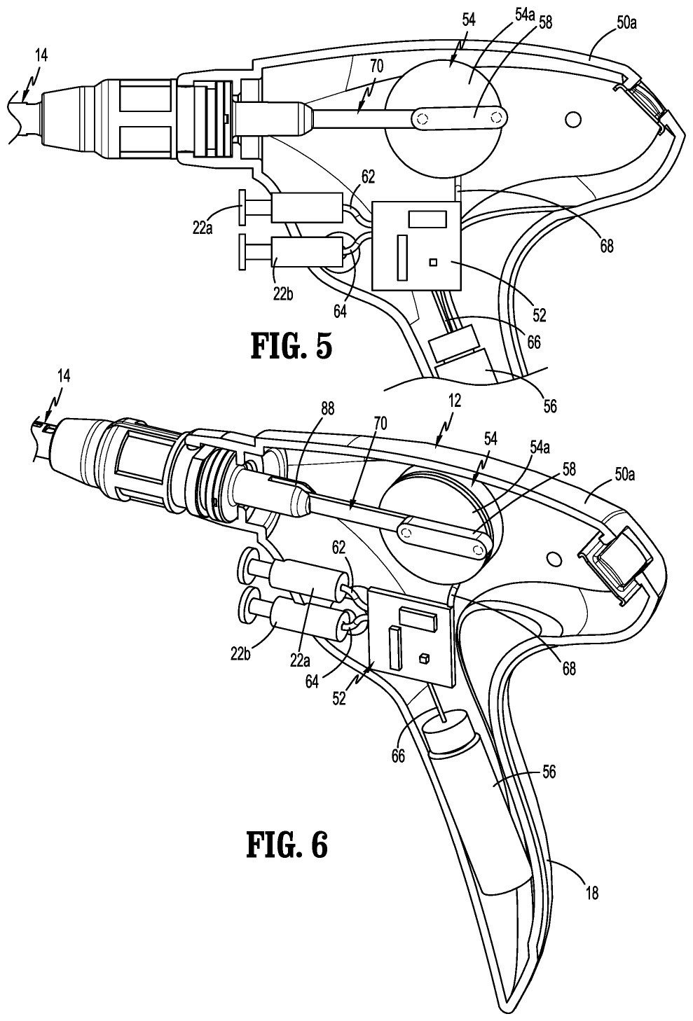

FIGS. 4-6 illustrate the handle assembly 12 which includes a housing 50 (FIG. 1), the actuation buttons 22a, 22b, a controller circuit 52, a motor 54, a battery 56, and a link or coupler 58. The housing 50 is formed from molded half-sections 50a and 50b which may be secured together using any known fastening substance or device such as adhesive or screws. The molded half-sections 50a, 50b define a cavity 60 that receives the other components of the handle assembly 12. The actuation buttons 22a, 22b are coupled to the controller circuit 52 by first and second wires 62 and 64, respectively. The controller circuit 52 includes one or more integrated circuits or chips and is coupled to a battery 56 by a third wire 66 and to the motor 54 by a fourth wire 68. The motor 54 includes a rotatable drum 54a. When one of the actuation buttons 22a, 22b is depressed, the motor 54 is actuated to perform a specific task as described in further detail below.

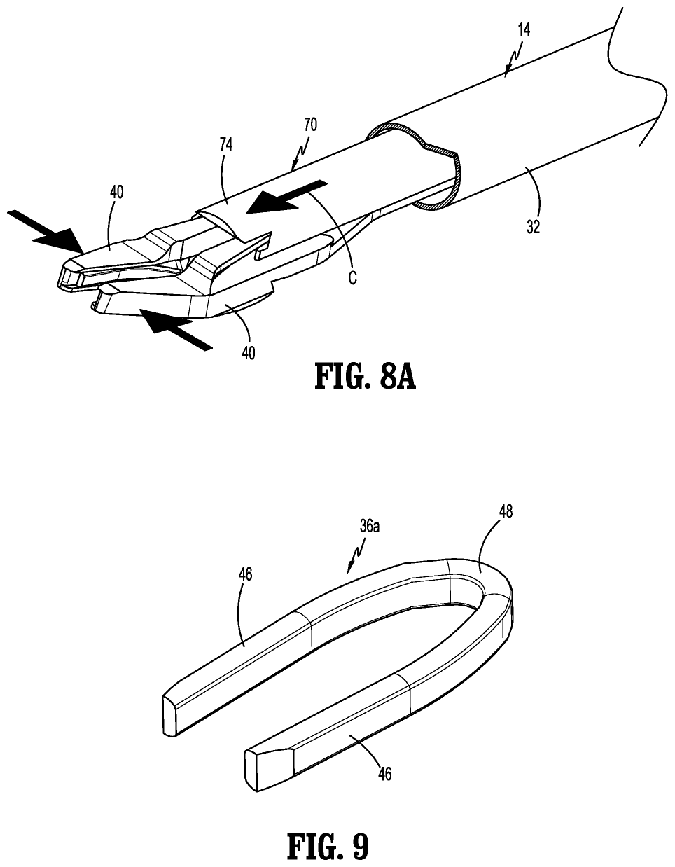

The elongate body 14 includes a drive member 70 that can move longitudinally within the outer tubular body 32 of the elongate body 14. The drive member 70 includes a proximal end 72 that extends into the cavity 60 of the housing 50 (FIG. 1) of the handle assembly 12 and a distal end 74 (FIG. 8A) that is engaged with the jaw assembly 16. The proximal end 72 of the drive member 70 is coupled to the rotatable drum 54a of the motor 54 by the link 58. When the rotatable drum 54a of the motor 54 is rotated, the link 58 translates the rotatable motion of the rotatable drum 54a into longitudinal movement of the drive member 70. In aspects of the disclosure, the link 58 includes pins 80 that are pivotably received in openings 82 in the drive member 70 and the rotatable drum 54a of the motor 54 to couple the link 58 to the drive member 70 and the motor 54. It is also envisioned that the link 58 can be coupled to the drive member 70 and the motor 54 using a variety of other techniques. The proximal portion 72 of the drive member 70 includes a slot 88 that allows the link 58 to pivot as the rotatable drum 54a of the motor 54 rotates.

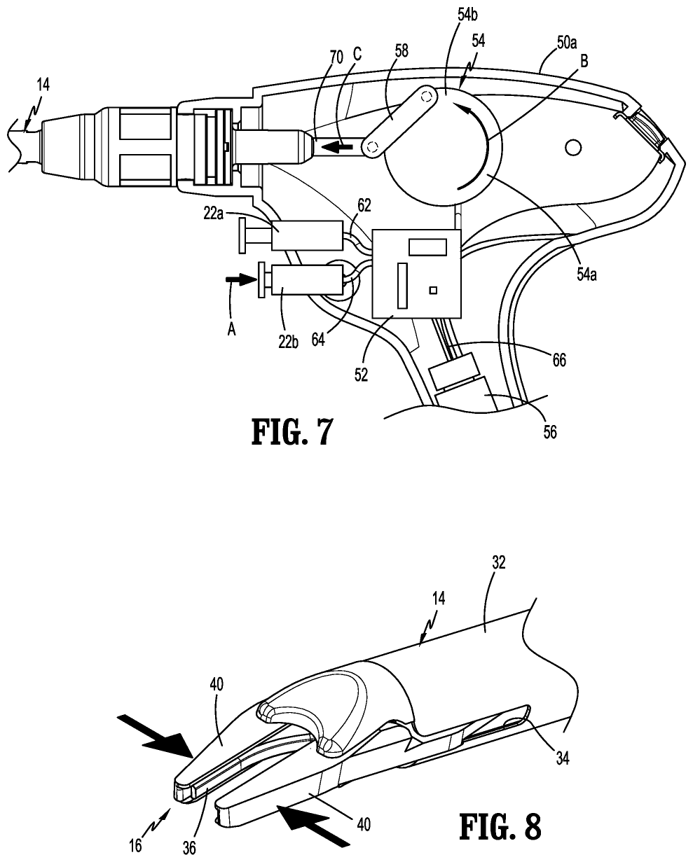

FIGS. 7-9 illustrate the clip applier 10 as the clip applier 10 is actuated to partially crimp a clip 36. When the actuation button 22b (FIG. 7) is depressed in the direction indicated by arrow “A” in FIG. 7, the motor 54 is energized or activated via the controller circuit 52 to partially crimp a clip 36a (FIG. 9). When the motor 54 is energized, the rotatable drum 54a of the motor 54 is rotated in the direction indicated by arrow “B” in FIG. 7 to advance the drive member 70 longitudinally in the direction indicated by arrows “C” in FIGS. 7 and 8A. As the drive member 70 is advanced, the distal portion 74 (FIG. 8A) of the drive member 70 cams the jaw members 40 towards each other in the direction indicated by arrows “D” in FIGS. 8 and 8A from the open position to a partially clamped position to partially crimp the clip 36a (FIG. 9). The controller circuit 52 controls operation of the motor 54 to limit longitudinal advancement of the drive member 70 to a predetermined first longitudinal position. As stated above, in the first longitudinal position of the drive member 70, the jaw members 40 are moved to a partially clamped position to partially crimp the clip 36a. It is noted that the circuit controller 52 can be configured to control longitudinal advancement of the drive member 70 to any desired longitudinal position to cause any desired degree of crimping of the clip 36a.

FIGS. 10-12 illustrate the clip applier 10 as the clip applier 10 is actuated to fully crimp a clip 36. When the actuation button 22a (FIG. 10) is depressed in the direction indicated by arrow “E” in FIG. 10, the motor 54 is energized via the controller circuit 52 to fully crimp a clip 36b (FIG. 12). When the motor 54 is energized, the rotatable drum 54a of the motor 54 is rotated in the direction indicated by arrow “F” in FIG. 10 to advance the drive member 70 longitudinally in the direction indicated by arrows “G” in FIG. 11A. As the drive member 70 is advanced, the distal portion 74 (FIG. 11A) of the drive member 70 cams the jaw members 40 towards each other in the direction indicated by arrows “H” in FIGS. 8 and 8A from the open position to a fully clamped position to fully crimp the clip 36b (FIG. 12). The controller circuit 52 controls operation of the motor 54 to limit longitudinal advancement of the drive member 70 to a predetermined second longitudinal position. As stated above, in the second longitudinal position of the drive member 70, the jaw members 40 are moved to a fully clamped position to fully crimp the clip 36b about tissue “T”.

Although only two positions are shown in this disclosure, the actuator 20 and the circuit controller 52 can be configured to advance the drive member 70 to multiple positons to provide multiple degrees of crimping of a respective clip 36. Each of these positions can be selected for use with a specific surgical procedure or for a tissue of a specified thickness.

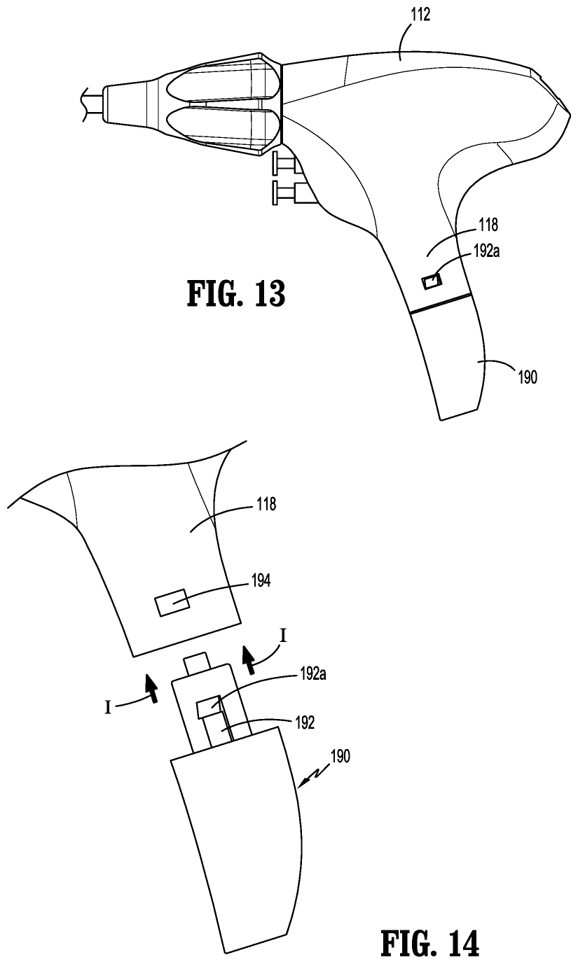

FIGS. 13 and 14 illustrate another aspect of the disclosure that includes a modification of the handle assembly 12 of the clip applier 10 shown generally as handle assembly 112. Handle assembly 112 is substantially similar to handle assembly 12 except that handle assembly 112 includes a battery pack 190 that is releasably coupled to the grip portion 118 of the handle assembly 112. In certain aspects of the disclosure, the grip portion 118 of the handle assembly 112 defines an opening 194 and the battery pack 190 includes a resilient finger 192 that includes a hook portion 192a. When the resilient finger 192 of the battery pack 190 is slid into the grip portion 118 of the handle assembly 112 in the direction indicated by arrow “I” in FIG. 14, the resilient finger 192 is deformed inwardly until the hook portion 192a becomes aligned with the opening 194. When this occurs, the hook portion 192a snaps outwardly into the opening 194 to secure the battery pack 190 to the grip portion 118.

Persons skilled in the art will understand that the devices and methods specifically described herein and illustrated in the accompanying drawings are non-limiting exemplary aspects of the disclosure. It is envisioned that the elements and features illustrated or described in connection with one exemplary embodiment may be combined with the elements and features of another without departing from the scope of the disclosure. As well, one skilled in the art will appreciate further features and advantages of the disclosure based on the above-described aspects of the disclosure. Accordingly, the disclosure is not to be limited by what has been particularly shown and described, except as indicated by the appended claims.

What is claimed is:

1. A surgical clip applier comprising:
a handle assembly including a housing defining a cavity, an actuator, and a motor, the motor positioned within the cavity of the housing;
an elongate body including an outer tubular housing and a drive member movable within the outer tubular housing, the drive member having a proximal portion extending into the cavity of the housing and a distal portion, the proximal portion coupled to the motor, the drive member movable from retracted position to an advanced position in response to activation of the motor; and
a jaw assembly including first and second jaw members that are configured to receive and support a clip, the first and second jaw members movable from an open position to a clamped position and being operably engaged with the distal portion of the drive member such that movement of the drive member from the retracted position to the advanced position moves the first and second jaw members from the open position to a fully clamped position, wherein the actuator is configured to activate the motor to selectively advance the drive member to a predetermined partially advanced position between the retracted and advanced positions to move the first and second jaw members to a partially clamped position between the open and clamped positions to partially crimp the clip positioned between the first and second jaw members.

2. The surgical clip applier of claim 1, wherein the handle assembly includes a link and the motor includes a rotatable drum, the drive member coupled to the rotatable drum of the motor by the link such that rotation of the rotatable drum causes movement of the drive member between the retracted and advanced positions.

3. The surgical clip applier of claim 1, wherein the handle assembly includes a controller circuit coupled to the motor and the actuator, the controller circuit configured to control operation of the motor to selectively advance the drive member to either the advanced position or the partially advanced position.

4. The surgical clip applier of claim 3, wherein the controller circuit includes one or more integrated circuits or chips.

5. The surgical clip applier of claim 4, wherein the handle assembly includes a battery that is coupled to the motor via the controller circuit.

6. The surgical clip applier of claim 5, wherein the housing of the handle assembly includes a grip portion and the battery is received in a battery pack that is releasably coupled to the grip portion.

7. The surgical clip applier of claim 1, wherein the actuator includes first and second actuation buttons coupled to the motor, the first actuator button being operable to activate the motor to move the drive member to the partially advanced position to partially crimp the clip and the second actuator button being operable to activate the motor to move the drive member to the advanced position to fully crimp the clip.

8. The surgical clip applier of claim 4, wherein the controller circuit is configured to control advancement of the drive member to any desired longitudinal position between the advanced and retracted positions to cause any desired degree of crimping of the clip.

9. The surgical clip applier of claim 1, wherein each of the first and second jaw members defines a channel, the channels receiving the clip.

10. The surgical clip applier of claim 1, further including a rotation knob supporting the elongate body, the rotation knob rotatably coupled to the housing of the handle assembly and being rotatable in relation to the handle assembly to facilitate rotation of the elongate body and jaw assembly in relation to the handle assembly.

11. A surgical clip applier comprising:
a handle assembly having an actuator and a motor;
an elongate body supported on the handle assembly, the elongate body including a drive member having a proximal portion and a distal portion, the proximal portion coupled to the motor;
a jaw assembly including first and second jaw members, the first and second jaw members being operably engaged with the drive member and movable in relation to each other between open and clamped positions in response to movement of the drive member; and
a clip supported between the first and second jaw members;
wherein the actuator includes a first actuator that is operable to move the drive member to a first position to move the first and second jaw members to a first jaw member position between the open and clamped positions to partially crimp a clip and a second actuator that is operable to advance the drive member to the advanced position to move the first and second jaw members to the clamped position to fully crimp the clip.

12. The surgical clip applier of claim 11, wherein the handle assembly includes a link and the motor includes a rotatable drum, the drive member coupled to the rotatable drum of the motor by the link such that rotation of the rotatable drum causes movement of the drive member between the retracted and advanced positions.

13. The surgical clip applier of claim 11, wherein the handle assembly includes a controller circuit coupled to the motor and the actuator, the controller circuit configured to control operation of the motor to selectively advance the drive member to either the advanced position or the partially advanced position.

14. The surgical clip applier of claim 13, wherein the controller circuit includes one or more integrated circuits or chips.

15. The surgical clip applier of claim 14, wherein the handle assembly includes a battery that is coupled to the motor via the controller circuit.

16. The surgical clip applier of claim 15, wherein the housing of the handle assembly includes a grip portion and the battery is received in a battery pack that is releasably coupled to the grip portion.

17. The surgical clip applier of claim 11, wherein each of the first and second jaw members defines a channel, the channels receiving the clip.

18. A surgical clip applier comprising:
a handle assembly including a housing defining a cavity, an actuator, a motor, a link, a controller circuit coupled to the actuator and the motor, and a battery coupled to the motor via the controller circuit, the motor and the controller circuit positioned within the cavity of the housing, wherein the actuator includes a first actuator and a second actuator;
an elongate body including an outer tubular housing and a drive member movable within the outer tubular housing, the drive member having a proximal portion extending into the cavity of the housing and a distal portion, the proximal portion coupled to the motor by the link, the drive member movable from retracted position to an advanced position in response to activation of the motor; and
a jaw assembly including first and second jaw members that are configured to receive and support a clip, the first and second jaw members movable in relation to each other from an open position to a fully clamped position and being operably engaged with the distal portion of the drive member such that movement of the drive member from the retracted position to the advanced position moves the first and second jaw members from the open position to the fully clamped position,
wherein the first actuator is operable to move the drive member to a first position to move the first and second jaw members to a first jaw member position between the open and clamped positions to partially crimp the clip and the second actuator that is operable to advance the drive member to the advanced position to move the first and second jaw members to the clamped position to fully crimp the clip.
